(19) United States
(12) Patent Application Publication
(10) Pub. No.: US 2022/0062288 A1
(43) Pub. Date: Mar. 3, 2022

(54) PHARMACEUTICAL COMPOSITIONS OF VIBEGRON FOR REDUCING BODY FAT

(71) Applicant: Jubilant Pharma Holdings Inc., Yardley, PA (US)

(72) Inventor: Indranil Nandi, Yardley, NJ (US)

(73) Assignee: Jubilant Pharma Holdings Inc., Yardley, PA (US)

(21) Appl. No.: 17/464,390

(22) Filed: Sep. 1, 2021

Related U.S. Application Data
(60) Provisional application No. 63/073,727, filed on Sep. 2, 2020.

(51) Int. Cl. A61K 31/519; A61K 9/00; A61K 47/10; A61K 47/02; A61K 47/12

(52) U.S. Cl. CPC A61K 31/519; A61K 9/0019; A61K 47/12; A61K 47/02; A61K 47/10

(57) ABSTRACT

The present invention relates to methods and compositions of vibegron or its pharmaceutically acceptable salts, esters, solvates, polymorphs, enantiomers, or mixtures thereof for reducing body fat. The present invention further provides a method of treating localized fat, double chin disorder, benign symmetric lipomatosis, adiposis dolorosa, lipedema, and familial partial lipodystrophy in a subject by administration of the parenteral pharmaceutical compositions of vibegron. It also relates to a process for the preparation of such compositions.

### FIELD OF THE INVENTION

The present invention relates to methods and compositions of vibegron or its pharmaceutically acceptable salts, esters, solvates, polymorphs, enantiomers, or mixtures thereof for reducing body fat. The present invention provides a method of treating double chin disorder, benign symmetric lipomatosis, adiposis dolorosa, lipedema, and familial partial lipodystrophy in a subject by administration of parenteral pharmaceutical compositions of vibegron. It also relates to a process for the preparation of such compositions.

### BACKGROUND OF THE INVENTION

Vibegron is an oral, once-daily, small molecule beta-3 adrenergic agonist useful for the treatment of patients with overactive bladder (OAB). Vibegron is chemically known as (6S)—N-[4-[[(2S,5R)-5-[(R)-hydroxy(phenyl)methyl]pyrrolidin-2-yl]methyl]phenyl]-4-oxo-7,8-dihydro-6H pyrrolo[1,2a]pyrimidine-6-carboxamide and is represented by the following formula:

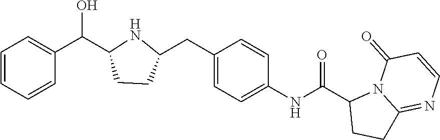 [SMILES: O=C(Nc1ccc(C[C@@H]2CC[C@H](C(O)c3ccccc3)N2)cc1)C1CCc2nccc(=O)n21]

Kyorin Pharmaceuticals and Urovant Sciences have obtained a license from Merck for the development of the vibegron dosage form. Vibegron 50 mg tablets (Beover®) has been approved in Japan for the treatment of overactive bladder. Vibegron 75 mg tablets (Gemtesa®) has been approved in the US for the treatment of overactive bladder. Vibegron is also under investigation by Urovant for two additional indications, the treatment of OAB in men with benign prostatic hyperplasia (BPH) and the treatment of pain associated with irritable bowel syndrome (IBS).

U.S. Pat. Nos. 8,399,480, 8,247,415, and 8,653,260 disclose vibegron as a product and its use in the treatment of disorders selected from overactive bladder, urinary incontinence, urge urinary incontinence, and urinary urgency.

PCT Publication No. 2018/224989 discloses a method of treating overactive bladder with oral administration of about 60 mg to about 90 mg of vibegron per day.

Excess body fat is a growing global health problem. It is a metabolic disorder related to internal appetite regulation and energy metabolism. It refers to a state wherein fat is over-accumulated in an individual and which may further lead to other health problems such as hypertension, diabetes, hyperlipidemia, cardiovascular diseases, and even cancer. According to the definition by the World Health Organization (WHO), individuals with a body mass index (BMI) greater than 25 are overweight and individuals with BMI greater than 30 are obese.

Anorexiant drugs are often used in the treatment of obesity. They act by suppressing the appetite in an individual. Examples of anorexiant drugs include caffeine, ephedrine, phenylpropanolamine, amphetamine, diethylpropion, mazindol, phentermine, and fluoxetine. However, treatment therapy with anorexiant drugs is associated with side effects.

Oral treatment options are not adequate for the reduction of localized fat, obesity, and obesity-related orphan conditions such as double chin disorder, benign symmetric lipomatosis, adiposis dolorosa, lipedema, and familial partial lipodystrophy. To reduce localized fat at specific locations (such as chin and face, waist, abdomen, legs, arms, etc.), the most prevalent technology is liposuction. However, the procedure of liposuction causes severe damage to the nerves, blood vessels, and other tissues. Liposuction also comes with the risk of infection, bleeding, bruising, swelling, and severe pain.

The current treatment options for obesity and localized fat have limited efficacy and are prone to clinically significant side effects. Accordingly, there is an unmet need for pharmaceutical compositions which can effectively reduce body fat and are useful in the treatment of localized fat reduction and conditions such as double chin disorder, benign symmetric lipomatosis, adiposis dolorosa, lipedema, and familial partial lipodystrophy.

Till date no β3-adrenergic receptor agonist is approved in the USA for the treatment of reduction of localized fat and conditions such as double chin disorder, benign symmetric lipomatosis, adiposis dolorosa, lipedema, and familial partial lipodystrophy. Vibegron, a beta-3 adrenergic agonist can be useful in the treatment of double chin disorder, benign symmetric lipomatosis, adiposis dolorosa, lipedema, and familial partial lipodystrophy.

The inventor of the present invention propose parenteral compositions of vibegron for the treatment of localized fat deposition, double chin disorder, benign symmetric lipomatosis, adiposis dolorosa, lipedema, and familial partial lipodystrophy. The compositions and method are useful to reduce fat non-surgically in a subject having localized fat deposits. The compositions are suitable for subcutaneous or intramuscular injection administration to an overweight or obese subject without the need of surgery. The parenteral compositions are expected to exhibit desired technical attributes such as pH, viscosity, drug release, stability, and sterility.

### SUMMARY OF THE INVENTION

The present invention relates to methods and compositions of vibegron or its pharmaceutically acceptable salts, esters, solvates, polymorphs, enantiomers, or mixtures thereof for reducing body fat. It provides a method of treating double chin disorder, benign symmetric lipomatosis, adiposis dolorosa, lipedema, and familial partial lipodystrophy in a subject by administration of parenteral pharmaceutical compositions of vibegron. It also relates to a process for the preparation of such compositions. The parenteral compositions of vibegron provide a suitable therapeutic option for the treatment of reduction of localized fat, double chin disorder, and related disorders.

### DETAILED DESCRIPTION OF THE INVENTION

The present invention can be more readily understood by the following detailed description of the invention.

The term “overweight”, as used herein, refers to an adult individual having a body mass index (BMI) greater than or equal to 24 and less than 27.

The term “obese”, as used herein, refers to an adult individual having a body mass index (BMI) of greater than or equal to 30.

The term “localized fat”, as used herein, refers to the fat at the chin (submental), face, waist, legs, breast, lower eyelids, neck, buttocks, arms, knees, peri-orbital region, intra-orbital region, abdomen, lipoma and other areas of localized fat in the body.

The term “dosage form”, as used herein, is intended to encompass a drug product comprising vibegron or its pharmaceutically acceptable salts, esters, solvates, polymorphs, stereoisomers, derivatives or mixtures thereof, and other inert ingredient(s) (pharmaceutically acceptable excipients). Such pharmaceutical dosage forms are synonymous with “formulation” and “composition”. The dosage form or the composition of the present invention refers to a parenteral composition or injectable composition or injection composition. The parenteral composition can be an injection solution, injection suspension, injection emulsion, injection dispersion, infusion solution, infusion suspension, infusion emulsion, infusion dispersion, powder for reconstitution, implants, and depot formulations such as depot injection solution, depot injection emulsion, and depot injection suspension. The composition is provided as a liquid composition or as a dry composition (lyophilized powder for reconstitution). The compositions can be immediate-release formulations, delayed-release formulations, extended-release formulations, sustained-release formulations, pulsatile release formulations, and mixed immediate release and controlled release formulations. Particularly, the composition of the present invention is an immediate release parenteral composition. The parenteral composition of vibegron may be provided as a single or multiple dose composition.

The term “parenteral administration”, as used herein, refers to the administration of the composition by injection into a body. Parenteral administration can include subcutaneous, intravenous, intra-arterial, intramuscular, and intradermal administration. Preferably, the composition is administered subcutaneously or intramuscularly.

The term “subject”, as used herein, refers to a human or non-human animal (e.g., a mammal, e.g., a dog, a cat, a monkey, a horse, etc.). The terms “subject”, “patient”, individual” and the like are used interchangeably herein. In certain non-limiting embodiments, the patient, subject, or individual is a human.

The terms “effective amount”, “pharmaceutically effective amount”, and “therapeutically effective amount”, as used herein, refers to non-toxic and sufficient amounts capable of providing desired biological results. The result may be a reduction and/or alleviation of the sign, symptom, or cause of the disease, or any other desired alteration of the biological system. The appropriate therapeutic amount in any individual case can be determined by one of ordinary skill in the art using routine testing.

The term “non-surgical” refers to a medical procedure that does not require an incision.

The term “vibegron”, as used herein, refers to (6S)—N-[4-[[(2S,5R)-5-[(R)-hydroxy(phenyl)methyl]pyrrolidin-2-yl]methyl]phenyl]-4-oxo-7,8-dihydro-6H pyrrolo [1,2a]pyrimidine-6-carboxamide, its free base, its pharmaceutically acceptable salts, esters, solvates, polymorphs, enantiomers, isomers, derivatives, and mixtures thereof. Vibegron in the parenteral composition is present in an amount from about 0.01% to about 95%, 0.01% to about 80%, particularly from about 0.01% to about 50%, particularly from about 0.01% to about 40%, particularly from about 0.01% to about 30%, particularly from about 0.01% to about 20%, particularly from about 0.01% to about 10%, particularly from about 0.01% to about 5%, particularly from about 0.01% to about 2%.

The term “pharmaceutically acceptable excipient” as used herein refers to a substance that is useful in preparing a pharmaceutical composition that is generally safe, non-toxic, pharmacologically inert, and is acceptable for veterinary or human pharmaceutical use.

The term “stable” as used herein, refers to chemical stability, wherein not more than 5% w/w of total related substances are formed on storage at 40° C.±2° C. and 75% relative humidity (R.H.) ±5% RH for a period of at least one month, for a period of at least three months, and more preferably for a period of at least six months.

The term “about” as used herein, means±approximately 10% of the indicated value, such that “about 10 percent” indicates approximately 08 to 12 percent.

The term “%” or “percent” of vibegron, carrier, and pharmaceutically acceptable excipients as used herein, refers to % w/w or % w/v of the parenteral composition.

The term “lyophilization” as used herein, refers to freeze-drying or dehydration technique which involves removing a solvent such as water, a water-miscible solvent, from vibegron composition typically by sublimation under high vacuum when the composition is in a frozen state.

The term “lyophilized composition”, as used herein refers to solid residue or powder which is produced or which remains after the lyophilization procedure.

An “immediate-release formulation” as used herein, refers to a formulation that has been formulated to allow vibegron to act as quickly as possible. An “extended-release formulation” refers to a pharmaceutical formulation that has been adapted such that the drug is released over an extended period of time.

A double chin is characterized by extra fat that develops beneath the chin. Benign symmetric lipomatosis (BSL) is a rare proliferative disorder of the adipose tissue, characterized by symmetrical fat deposits, predominantly in the neck and shoulder area, upper back, and arms. Adiposis dolorosa is a rare condition characterized by the growth of multiple, painful, lipomas (benign, fatty tumors). The lipomas may occur anywhere on the body and can cause severe pain. Other symptoms may include weakness, fatigue, and memory disturbances. It usually occurs in adults, and women are more commonly affected than men. Lipedema is a disorder characterized by symmetric enlargement of the legs due to deposits of fat beneath the skin. It is a common condition, occurring almost exclusively in women (affecting up to 11% of women). Familial partial lipodystrophy is a group of rare genetic lipodystrophic syndromes characterized (also known as Kobberling lipodystrophy/Dunnigan lipodystrophy) by fat loss from the limbs and buttocks, from childhood or early adulthood, and often associated with acanthosis nigricans, insulin resistance, diabetes, hypertriglyceridemia, and liver steatosis.

A first aspect of the present invention provides a method for treating a condition selected from the group comprising double chin disorder, benign symmetric lipomatosis, adiposis dolorosa, lipedema, familial partial lipodystrophy, and localized fat by administering a therapeutically effective amount of vibegron or its pharmaceutically acceptable salts, esters, solvates, polymorphs, enantiomers or mixtures thereof.

A second aspect of the present invention provides a pharmaceutical parenteral composition of vibegron or its pharmaceutically acceptable salts, esters, solvates, polymorphs, enantiomers, or mixtures thereof.

A third aspect of the present invention provides a pharmaceutical composition for parenteral administration comprising vibegron and at least one or more pharmaceutically acceptable excipients.

A fourth aspect of the present invention provides a pharmaceutical composition for parenteral administration comprising:
(a) vibegron or its pharmaceutically acceptable salts, esters, solvates, polymorphs, enantiomers, or mixtures thereof,
(b) a pharmaceutically acceptable carrier, and
(c) one or more pharmaceutically acceptable excipients,
   wherein the composition has a pH of about 3 to about 9.5.

According to an embodiment of the above aspect, the pharmaceutical composition for parenteral administration comprises:
(a) about 0.01% to about 40% of vibegron or its pharmaceutically acceptable salts, esters, solvates, polymorphs, enantiomers, or mixtures thereof,
(b) about 20% to about 99.99% of a pharmaceutically acceptable carrier, and
(c) one or more pharmaceutically acceptable excipients.

Another aspect of the present invention provides a pharmaceutical composition for parenteral administration comprising:
(a) vibegron or its pharmaceutically acceptable salts, esters, solvates, polymorphs, enantiomers, or mixtures thereof,
(b) a pH adjusting agent, and
(c) a carrier.

Another aspect of the present invention provides a pharmaceutical composition for parenteral administration comprising:
(a) vibegron or its pharmaceutically acceptable salts, esters, solvates, polymorphs, enantiomers, or mixtures thereof,
(b) a pH adjusting agent,
(c) a tonicity adjusting agent, and
(d) a carrier.

Another aspect of the present invention provides a pharmaceutical composition for parenteral administration comprising:
(a) vibegron or its pharmaceutically acceptable salts, esters, solvates, polymorphs, enantiomers, or mixtures thereof,
(b) a preservative,
(c) a pH adjusting agent,
(d) a tonicity adjusting agent,
(e) optionally an antioxidant, solvent, a chelating agent and a wetting agent, and
(f) a carrier.

Another aspect of the present invention provides a pharmaceutical composition for parenteral administration comprising:
(a) vibegron or its pharmaceutically acceptable salts, esters, solvates, polymorphs, enantiomers, or mixtures thereof,
(b) a preservative,
(c) a pH adjusting agent,
(d) a tonicity adjusting agent,
(e) a chelating agent,
(f) optionally an antioxidant and a wetting agent, and
(g) a carrier.

Another aspect of the present invention provides a pharmaceutical composition for parenteral administration comprising:
(a) about 0.01% to about 40% of vibegron or its pharmaceutically acceptable salts, esters, solvates, polymorphs, enantiomers, or mixtures thereof,
(b) about 0.01% to about 7% of preservative,
(c) about 0.01% to about 8% of a pH adjusting agent,
(d) about 0.1% to about 40% of a tonicity adjusting agent,
(e) optionally about 0.01% to about 7% of an antioxidant, about 0.01% to about 4% of a chelating agent and about 0.01% to about 2% of a wetting agent, and
(f) about 20% to about 99.99% of a pharmaceutically acceptable carrier.

Another aspect of the present invention provides a pharmaceutical composition for parenteral administration comprising:
(a) about 0.01% to about 40% of vibegron or its pharmaceutically acceptable salts, esters, solvates, polymorphs, enantiomers, or mixtures thereof,
(b) about 0.01% to about 7% of a preservative,
(c) about 0.01% to about 8% of a pH adjusting agent,
(d) about 0.1% to about 40% of a tonicity adjusting agent,
(e) about 0.01% to about 2% of a wetting agent,
(f) optionally about 0.01% to about 7% of an antioxidant and about 0.01% to about 4% of a chelating agent, and
(g) about 20% to about 99.99% of a pharmaceutically acceptable carrier.

Another aspect of the present invention provides a lyophilized powder for reconstitution for parenteral administration comprising vibegron or its pharmaceutically acceptable salts, esters, solvates, polymorphs, enantiomers or mixtures thereof and at least one or more pharmaceutically acceptable excipients.

Another aspect of the present invention provides a lyophilized powder for reconstitution for parenteral administration comprising:
(a) vibegron or its pharmaceutically acceptable salts, esters, solvates, polymorphs, enantiomers, or mixtures thereof,
(b) a preservative,
(c) a pH adjusting agent,
(d) a tonicity adjusting agent, and
(e) optionally an antioxidant, a chelating agent, and a wetting agent.

Another aspect of the present invention provides a kit comprising:
(a) a first container comprising a parenteral composition of vibegron or its pharmaceutically acceptable salts, esters, solvates, polymorphs, enantiomers, or mixtures thereof, and
(b) a delivery device capable of delivering the composition. The specific type of delivery device is not particularly limited but may be a syringe, pen, and auto-injector.

Another aspect of the present invention provides a kit comprising:
(a) a parenteral composition of vibegron or its pharmaceutically acceptable salts, esters, solvates, polymorphs, enantiomers, or mixtures thereof in a suitable container such as a vial, ampoule, syringe, auto-injector, pen (single or multi-compartment),
(b) optionally a container comprising a carrier for preparing the composition, and
(c) instructions for preparing and administering the composition.

The parenteral composition of the present invention has a pH of about 3 to about 9.5, particularly a pH of about 3 to about 8, particularly a pH of about 4 to about 8, and particularly a pH of about 5 to about 7.

According to another embodiment, the parenteral composition of vibegron or its pharmaceutically acceptable salts, esters, solvates, polymorphs, enantiomers, or mixtures thereof is free of preservative.

According to another embodiment, the parenteral composition of vibegron or its pharmaceutically acceptable salts, esters, solvates, polymorphs, enantiomers, or mixtures thereof is free of antioxidant.

According to another embodiment, the parenteral composition of vibegron or its pharmaceutically acceptable salts, esters, solvates, polymorphs, enantiomers, or mixtures thereof is free of preservative and antioxidant.

According to another embodiment, the parenteral composition of vibegron or its pharmaceutically acceptable salts, esters, solvates, polymorphs, enantiomers, or mixtures thereof is stable upon storage.

According to another embodiment, the parenteral composition of vibegron or its pharmaceutically acceptable salts, esters, solvates, polymorphs, enantiomers, or mixtures thereof is stable upon autoclaving for 20 minutes at 121° C. and 2 bar.

According to another embodiment, the parenteral composition of vibegron or its pharmaceutically acceptable salts, esters, solvates, polymorphs, enantiomers, or mixtures thereof is free of microbial content during storage.

Another aspect of the present invention provides a pharmaceutical composition for use in the treatment of double chin disorder, benign symmetric lipomatosis, adiposis dolorosa, lipedema, familial partial lipodystrophy, and localized fat in a subject comprising parenteral administration to the subject comprising:
(a) vibegron or its pharmaceutically acceptable salts, esters, solvates, polymorphs, enantiomers, or mixtures thereof,
(b) a pharmaceutically acceptable carrier, and
(c) one or more pharmaceutically acceptable excipients.

Another aspect of the present invention provides a pharmaceutical composition for use in the treatment of double chin disorder, benign symmetric lipomatosis, adiposis dolorosa, lipedema, familial partial lipodystrophy, and localized fat in a subject comprising parenteral administration to the subject comprising:
(a) about 0.01% to about 40% of vibegron,
(b) about 20% to about 99.99% of a pharmaceutically acceptable carrier, and
(c) one or more pharmaceutically acceptable excipients.

Another aspect of the present invention provides a method for treating double chin disorder, benign symmetric lipomatosis, adiposis dolorosa, lipedema, familial partial lipodystrophy, and localized fat in a subject comprising parenteral administration of a composition comprising an effective amount of vibegron, wherein the composition comprises:
(a) about 0.01% to about 40% of vibegron or its pharmaceutically acceptable salts, esters, solvates, polymorphs, enantiomers, or mixtures thereof,
(b) about 20% to about 99.99% of a pharmaceutically acceptable carrier, and
(c) one or more pharmaceutically acceptable excipients.

Another aspect of the present invention provides a pharmaceutical composition for parenteral administration of vibegron or its pharmaceutically acceptable salts, esters, solvates, polymorphs, enantiomers, or mixtures thereof for reducing localized fat.

Another aspect of the present invention provides a method for treating a condition selected from the group comprising double chin disorder, benign symmetric lipomatosis, adiposis dolorosa, lipedema, and familial partial lipodystrophy by administering a parenteral composition comprising a therapeutically effective amount of vibegron or its pharmaceutically acceptable salts, esters, solvates, polymorphs, enantiomers or mixtures thereof.

According to an embodiment of the above aspects, the parenteral administration is by subcutaneous injection.

According to another embodiment of the above aspects, the parenteral administration is by intramuscular injection.

According to another embodiment of the above aspects, the parenteral administration is by subcutaneous injection or intramuscular injection.

According to another embodiment of the above aspects, the parenteral composition is an immediate release composition of vibegron or its pharmaceutically acceptable salts, esters, solvates, polymorphs, enantiomers, or mixtures thereof.

According to another embodiment, the parenteral composition can be injected directly into a treatment site of a patient in need of fat removal without surgery. The composition is administered directly to the subcutaneous fat layer (tissue).

According to another embodiment, the parenteral composition can be injected into the abdomen, chin, waist, arms, legs, knees, thigh, chest, breast, neck, face, buttocks, peri-orbital region, intra-orbital region, or to a particular fat deposit area.

According to another embodiment of the above aspects, one or more pharmaceutically acceptable excipients are selected from the group comprising preservatives, viscosity adjusting agents, diluents, solvents, tonicity adjusting agents, pH adjusting agents, chelating agents, wetting agents, and anti-oxidants.

Another aspect of the present invention provides the use of therapeutically effective amounts of parenteral compositions of vibegron or its pharmaceutically acceptable salts, esters, solvates, polymorphs, enantiomers or mixtures thereof in the manufacture of a medicament for treating obesity, and its related metabolic disorders, hypertension, diabetes, patients at risk of having diabetes (pre-diabetes), cardiovascular diseases, hyperglycaemia, gallbladder diseases, excess fat on the chin (submental fullness or double chin disorder), binge eating, hypothyroidism, excess fat on the breast, adiposis dolorosa, benign symmetric lipomatosis, lipedema, familial lipodystrophy, familial partial lipodystrophy, HIV lipodystrophy, Bardet-Biedl syndrome, hypertrophy of dorsocervical fat/dorsocervical fat hypertrophy (“buffalo hump”), lipoma, lipomatosis, moon facies, Down syndrome, pseudo-Cushing syndrome, Cohen syndrome, Cushing syndrome, Prader-Willi syndrome, Turner syndrome, or Madelung disease.

According to another embodiment, vibegron or its pharmaceutically acceptable salts, esters, solvates, polymorphs, enantiomers or mixtures thereof is present in the parenteral composition in an amount from about 0.01% to about 95%, particularly from about 0.01% to about 80%, particularly from about 0.01% to about 50%, particularly from about 0.01% to about 40%, particularly from about 0.01% to about 30%, particularly from about 0.01% to about 20%, particularly from about 0.01% to about 10%, particularly from about 0.01% to about 5%.

According to another embodiment, the pharmaceutical composition for parenteral administration is administered twice daily, once daily, once weekly, once bi-monthly, once in three weeks, once monthly, once in two months, once in three months, or once in six months.

According to another embodiment, the pharmaceutical composition for parenteral administration is administered for at least 1 day, 3 days, 1 week, at least 2 weeks, at least 4 weeks, at least 8 weeks, at least 12 weeks, at least 24 weeks, or at least 28 weeks.

Another aspect of the present invention provides a process for the preparation of a pharmaceutical composition for parenteral administration of vibegron comprising the steps of:
(a) preparing a mixture comprising vibegron or its pharmaceutically acceptable salts, esters, solvates, polymorphs, enantiomers or mixtures thereof, carrier, and one or more pharmaceutically acceptable excipients.

According to an embodiment, the process comprises one or more additional steps selected from the group comprising of:
(b) purging the mixture (a) with an inert gas, and/or
(c) filtering the mixture through a filter e.g. with a filter having an average pore size of not more than 0.5 μm; and/or
(d) filtering the mixture into a suitable container e.g. ampoule or vial, and/or
(e) autoclaving the mixture at elevated temperature and elevated pressure e.g. at 121° C. and 2 bar for at least 20 minutes.

Another embodiment relates to a process for preparing a parenteral composition comprising: a) preparing a first sterile solution comprising one or more pharmaceutically acceptable excipients; b) preparing a second sterile solution comprising vibegron or its pharmaceutically acceptable salts, esters, solvates, polymorphs, enantiomers or mixtures thereof; c) dispersing both solutions to form the final sterile composition.

Another embodiment relates to a process for preparing a parenteral composition comprising: a) dissolving vibegron or its pharmaceutically acceptable salts, esters, solvates, polymorphs, enantiomers, or mixtures thereof in a solvent; b) charging the drug solution in an another suitable solvent, cooling below an appropriate temperature, and c) filtering the particles of the drug, thus obtaining sterile particles.

Another aspect of the present invention provides a process for the preparation of a lyophilized powder composition of vibegron comprising the steps of lyophilizing a mixture comprising vibegron or its pharmaceutically acceptable salts, esters, solvates, polymorphs, enantiomers, or mixtures thereof and one or more pharmaceutically acceptable excipients.

Another aspect of the present invention provides a process for the preparation of a lyophilized powder composition of vibegron, wherein the process comprises the steps of: dissolving vibegron, one or more pharmaceutically acceptable excipients, and optional buffer in a solvent; filtering to eliminate any bacteria or other contaminants; sub-packaging into sterile containers; lyophilizing; and drying.

According to another embodiment, methods used for the preparation of the parenteral composition may be selected from high shear homogenization, high-pressure homogenization, ultrasonication/high-speed homogenization, admixture of solvents, solubilizers, and active to prepare a suspension, admixture of active, carrier and one or more excipients, solvent emulsification, evaporation, lyophilization, and the like.

According to another embodiment, the parenteral composition of vibegron includes particle size of vibegron, having a particle size distribution such that D₉₀ is less than about 200 μm, D₅₀ is less than about 100 μm and D₁₀ is less than about 50 μm. Preferably, the particle size distribution is D₉₀ is less than about 100 μm, D₅₀ is less than about 70 μm and D₁₀ is less than about 30 μm. The terms D₁₀, D₅₀, and D₉₀ used herein as per the present invention indicate that 10%, 50%, and 90% of the distribution is below this value. The particle size of vibegron can be measured by suitable techniques such as Laser light scattering (e.g. Malvern Light Scattering), Coulter counter, microscopy, and any other technique known in the art.

A suitable volume of the composition of the present invention for injection is in the range of about 0.1 mL to about 100 mL, about 0.1 mL to about 50 mL, about 0.1 mL to about 20 mL, about 0.1 mL to about 10 mL, about 0.1 mL to about 2 mL, and 0.1 mL to about 1 mL.

The composition of the present invention comprises pharmaceutically acceptable excipients selected from the group comprising preservatives, viscosity adjusting agents, diluents, solvents, tonicity adjusting agents, pH adjusting agents, chelating agents, wetting agents, and anti-oxidants.

Suitable preservatives are selected from the group comprising benzyl alcohol, chlorobutanol, 2-ethoxyethanol, m-cresol, chlorocresol, benzalkonium chloride, benzethonium chloride, benzoic acid, sorbic acid, chlorhexidine, thimerosal, methyl paraben, propyl paraben, phenyl mercuric acetate, phenyl mercuric nitrate, and combinations thereof. The preservative is present in an amount ranging from about 0.01% to about 15%, particularly from about 0.01% to about 7%, particularly from about 0.1% to about 5%.

Suitable pH adjusting agents or buffering agents are selected from the group comprising inorganic acids and organic acids e.g. hydrochloric acid, acetic acid, adipic acid, ascorbic acid, sodium ascorbate, sodium ethoxide, malic acid, succinic acid, tartaric acid, fumaric acid, and citric acid; inorganic bases e.g. sodium hydroxide, potassium hydroxide, sodium carbonate, sodium hydrogen carbonate, magnesium carbonate, calcium carbonate, magnesium oxide, ammonia; organic bases e.g. basic amino acid such as lysine, arginine; borate buffers, tartarate buffers, lactate buffers, citrate buffers, phosphate buffers (e.g. monosodium phosphate monohydrate and dibasic sodium phosphate anhydrous), citric acid/phosphate buffers, carbonate/carbonic acid buffers, succinate/succinic acid buffers, ammonium buffers, and combinations thereof. The pH adjusting agent or buffering agent is present in an amount ranging from about 0.01% to about 35%, particularly from about 0.01% to about 15%, particularly from about 0.01% to about 8%, particularly from about 0.1% to about 5%.

Suitable viscosity adjusting agents or suspending agents are selected from the group comprising povidone, polyvinylpyrrolidone compounds, and polyethylene glycols, cellulose derivatives e.g. methylcellulose, carboxymethylcellulose, sodium carboxymethylcellulose, hydroxyethylcellulose and hydroxypropylmethylcellulose, gums e.g. xanthan gum, acacia, alginic acid, alginates, carbomers, and combinations thereof. The viscosity adjusting agent or suspending agent is present in an amount ranging from about 0.1% to about 20%, particularly from about 0.1% to about 15%, particularly from about 0.5% to about 5%.

Suitable tonicity adjusting agents are selected from the group comprising sodium chloride, sodium sulfate, dextrose, mannitol, sorbitol, glycerol, potassium chloride, glycerin, lactose, trehalose, ammonium carbonate, ammonium chloride, ammonium lactate, ammonium nitrate, ammonium phosphate, ammonium sulfate, ascorbic acid, bismuth sodium tartrate, boric acid, calcium chloride, disodium calcium edetate, calcium gluconate, calcium lactate, citric acid, dextrose, diethanolamine, dimethyl sulfoxide, disodium edetate, trisodium edetate monohydrate, sodium fluorescein, fructose, galactose, lactic acid, lactose, magnesium chloride, magnesium sulfate, polyethylene glycol, potassium acetate, potassium chlorate, potassium chloride, potassium iodide, potassium nitrate, potassium phosphate, potassium sulfate, propylene glycol, sodium acetate, sodium bicarbonate, sodium biphosphate, sodium bisulfate, sodium borate, sodium bromide, sodium cacodylate, sodium carbonate, sodium chloride, sodium citrate, sodium iodide, sodium lactate, metabisulfate sodium sulfite, sodium nitrate, sodium nitrite, sodium phosphate, sodium propionate, sodium succinate, sodium sulfite, sodium tartrate, sodium thiosulfate, sorbitol, maltose, sucrose, tartaric acid, triethanolamine, urea, urethane, uridine zinc sulfate, zinc chloride, albumin, amino acid, and combinations thereof. The tonicity adjusting agent is present in an amount ranging from about 0.1% to about 40%, particularly from about 0.1% to about 30%, particularly from about 0.5% to about 10%, particularly from about 0.5% to about 20%.

Suitable chelating agents are ethylenediamine tetracetic acid salts (e.g. edetate disodium), diethylenetriaminepentaacetic acid (DTPA), ethylene glycol-bis(β-aminoethyl ether)-tetraacetic acid (EGTA), N-(hydroxy ethyl) ethylenediaminetriacetic acid (HEDTA), 8-hydroxyquinoline, citric acid, tartaric acid, phosphoric acid, gluconic acid, and combinations thereof. The chelating agent is present in an amount ranging from about 0.01% to about 10%, particularly from about 0.01% to about 4%, particularly from about 0.01% to about 1%.

Suitable antioxidants are selected from the group comprising ascorbic acid and its derivatives e.g. sodium ascorbate; thiol derivatives e.g. thioglycerol, cysteine, acetylcysteine, cystine, dithioerythreitol, dithiothreitol, gluthathione, tocopherols, butylated hydroxyanisole, butylated hydroxytoluene, sulfurous acid salts e.g. sodium sulfate, sodium bisulfate, sodium bisulfite, sodium sulphite, sodium formaldehyde, and sodium thiosulfate, propyl gallate, and combinations thereof. The antioxidant is present in an amount ranging from about 0.01% to about 20%, particularly from about 0.01% to about 7%.

Suitable wetting agents or surfactants are selected from the group comprising anionic, cationic, zwitterionic, and non-ionic surfactants. Non-ionic surfactants may comprise polyoxyethylene fatty alcohol esters, sorbitan fatty acid esters (Spans), polyoxyethylene sorbitan fatty acid esters (e.g. polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g. Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL), PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate. The wetting agent or surfactant is present in an amount ranging from about 0.01% to about 30%, particularly from about 0.01% to about 10%, particularly from about 0.01% to about 5%, particularly from about 0.01% to about 2%, more particularly 0.01% to about 0.3%.

The carrier can be aqueous, water miscible solvents, or non-aqueous vehicle or a mixture of aqueous and non-aqueous vehicles e.g. water, alcohols such as ethanol, isopropanol, polyols such as propylene glycol, polyethylene glycols (e.g. polyethylene glycol 300, polyethylene glycol 400, polyethylene glycol 600, or polyethylene glycol 1000), glycerol, glycerin, vegetable oils such as soybean oil, castor oil, olive oil, saline solution, and combinations thereof. The carrier is present in an amount ranging from about 5% to about 99.99%, particularly from 10% to about 99.99%, particularly from 20% to about 99.99%, particularly from 30% to about 99.99%, particularly from 40% to about 99.99%.

Suitable diluents are selected from mannitol, glycine, lactose, sucrose, trehalose, sucralose, isomaltose, maltose, dextrose, arginine, dextran, hydroxyethyl starch, ficoll, gelatin, and combinations thereof. The diluent is present in an amount ranging from about 5% to about 99.99% of the powder composition.

Solvents used in the present invention are non-toxic, biocompatible and are selected from oils, C₂-C₆ aliphatic alcohols, ethanol, 1-propanol, 2-propanol, 1-butanol, 2-butanol, 1-pentanol, 2-pentanol, 3-pentanol, isopropanol, benzyl alcohol, glycol ethers (e.g., including, but limited to, diethyleneglycol monoethyl ether (DGME, Transcutol®), butyl diglycol, dipropylene glycol n-butyl ether, ethyleneglycol monoethyl ether, dipropylene glycol monomethyl ether, propylene glycol monomethyl ether, propylene glycol monoethyl ether, liquid polyethylene glycols (PEGs) (PEG 200, PEG 300, PEG 400), carbonates (e.g. propylene carbonate), 2-pyrrolidone, N-methylpyrrolidone, dimethyl isosorbide, dimethylacetamide, glycerol formal, dichloromethane, chloroform, ethyl acetate, dioxane, ethyl ether, acetone, tetrahydrofuran, benzene, toluene, glacial acetic acid, petroleum ether, alkane, paraffine, dimethylsulfoxide, liquid polyethylene glycol, block copolymers of oxyethylene, polyoxyethylene alcohol, polyoxyethylene fatty acid esters, hydrocarbons, n-propane, n-butane, isobutane, n-pentane, iso-pentane, neo-pentane, n-hexane, ethers, diethyl ether, hydroxylated solvents, dextrose, aqueous saline, water, purified water, diethylene glycol ethyl ether, isopropylidene glycerol, glycerol, N-methyl-pyrrolidone, N-pyrrolidone, methylethylketone, 1-dodecylazacycloheptane, dipropyleneglycol methyl ether, methyl acetate, ethyl lactate, dimethylformamide, N,N-diethyl-m-toluamide, ethylacetamide, caprolactam, decylmethylsulfoxide, triacetin and the like and mixtures thereof. The solvent is present in an amount ranging from about 0.1% to about 95%.

In another embodiment, the parenteral compositions are sterilized by dry heat, moist heat, gaseous, filtration, irradiation, terminal sterilization, and other methods known in the art. According to another embodiment, the parenteral composition is in concentrated form which can be diluted with an appropriate medium e.g. saline.

The composition of the present invention may be contained in any pharmaceutically acceptable container e.g., ampules, vials, injection pen, cartridge.

Actual dosage levels of the active ingredients in the compositions of the present invention may be varied so as to obtain an amount of the active ingredient which is effective to achieve the desired therapeutic or prophylactic response for a particular subject.

The pharmaceutical parenteral compositions of the present invention comprise vibegron from about 0.05 μg to about 3500 mg per day. In some embodiments, the dose of vibegron ranges from about 0.1 μg to about 800 mg per day, from about 0.1 μg to about 700 mg per day, about 0.1 μg to about 600 mg per day, about 0.1 μg to about 500 mg per day, about 0.1 μg to about 400 mg per day, about 0.1 μg to about 300 mg per day, about 0.1 μg to about 200 mg per day, about 0.1 μg to about 100 mg per day, about 0.1 μg to about 50 mg per day, about 0.1 μg to about 20 mg per day, about 0.1 μg to about 10 mg per day, about 0.1 μg to about 5 mg per day, about 0.1 μg to about 4 mg per day, about 0.1 μg to about 3 mg per day, about 50 mg to 100 mg per day.

The compositions of the invention are administered for a time and in an amount sufficient to treat obesity, double chin disorder, benign symmetric lipomatosis, adiposis dolorosa, lipedema, and familial partial lipodystrophy, and localized fat reduction.

The compositions of the invention are administered within a plurality of treatment sessions.

The composition of the present invention may be administered by administering to multiple target sites (point) at regular intervals in the affected area with a single administration, and the total amount may refer to the total amount of the dose administered through these multiple target sites at the single administration. The target site may be set in a range of 1 to 60 for one affected area. In addition, the composition or preparation of the present invention comprises administration to one target site on one affected area at the single administration, and the total amount is calculated on the basis of the amount for the one target site.

The compositions of the present invention can also be lyophilized using lyophilization techniques known in the art and stored as a powder which can be reconstituted prior to administration. Typically, lyophilization is carried out in lyophilization equipment (a lyophilizer), which comprises a drying chamber with variable temperature controls, a condenser to collect water, and a vacuum system to reduce the pressure in the drying chamber.

Prior to administering the dry composition to a patient in need thereof, the dry or lyophilized composition is reconstituted. Reconstitution can take place in the container in which the dry composition of vibegron is provided such as in a vial, syringe, dual-chamber syringe, ampoule, and cartridge. Reconstitution is done by adding a predefined amount of reconstitution liquid or carrier to the dry composition. Reconstitution liquid or carrier is a sterile liquid, such as water or buffer, which may contain further additives, such as preservatives. Particularly, the reconstitution liquid is sterile water.

In a further embodiment, the vibegron is released from a depot. In an embodiment, vibegron is fully contained in a depot, typically a polymer gel, such as a hydrogel. In another embodiment, the active agent may be incorporated into microspheres, microcapsules, nanoparticles, liposomes, or the like for extended release.

The parenteral compositions of the present invention are expected to exhibit desired technical characteristics based on tests such as 1) pH: pH is measured by using a pH meter. 2) Sterility Test: It can be carried out by inoculation of a culture medium with the composition. If there is no evidence of microbial growth, then the preparation being examined passes the test for sterility. 3) Leakage test: Leakage test is employed to test the package integrity. Leakage test can be done by dye bath test. The test container is immersed in a dye bath. Vacuum and pressure are applied for some time. The container is removed from the dye bath and washed. The container is then inspected for the presence of dye either visually or by means of analytical techniques such as UV spectroscopy. 4) Pyrogen test. 5) Content uniformity. 6) Viscosity. 7) Clarity. 8). Drug release and 9) Stability.

Experimental tests for the effect of vibegron in reducing fat deposition in a subject in need thereof include Pre-clinical studies including: 1) In-vitro tests (In-vitro study of mouse preadipocyte viability, preadipocyte differentiation, preadipocyte apoptosis, and adipocyte apoptosis). 2) Animal study (effect of vibegron on inguinal lateral fat pad of hamsters/rats). Clinical studies including: 1) In vivo human study; 2) Body mass index (BMI) study (Quantitative methods for the analysis of weight loss or maintenance include measurements of body mass index (BMI). In some embodiments, BMI may be monitored in the present invention by determining a subject's body mass and height and then dividing the individual's body mass by the square of their height, with the value given in units of kg/m. BMI values may range from underweight to obesity and may be used to assess how much a subject's body weight departs from what is normal or desirable for a person of his or her height).

The following vibegron parenteral compositions are expected to exhibit desired technical attributes in the treatment of reducing body fat as given in examples, under sterile conditions as per the processes as mentioned in the present invention and are filled into a suitable container.

### EXAMPLES

### Examples 1-5. Vibegron Parenteral Injections

| | | Quantity (%) | | | | |
|---|---|---|---|---|---|---|
| Ingredients | Function | Example 1 | Example 2 | Example 3 | Example 4 | Example 5 |
| Vibegron | Active ingredient | 0.01-70 | 0.01-40 | 0.01-40 | 0.01-40 | 5 |
| Sodium | pH adjusting agent/ | 0.01-35 | 0.1-6 | 0.1-6 | 0.1-6 | 0.5 |
| phosphate | buffering agent | | | | | |
| Sodium | Tonicity adjusting | 0-40 | — | 0-40 | 0-2 | 1 |
| chloride | agent | | | | | |
| Benzyl alcohol | Preservative | — | — | 0-15 | 0.02-1.5 | 0.05 |
| Water | Carrier | q.s. | q.s. | q.s. | q.s. | q.s. |

TABLE 2
Examples 6-9

| | Quantity (%) | | | |
|---|---|---|---|---|
| Ingredients | 6 | 7 | 8 | 9 |
| Vibegron | 1 | 1 | 5 | 10 |
| Sodium acetate | — | — | 0.5 | 0.5 |
| Sodium phosphate monobasic | — | 0.2 | — | — |
| Sodium phosphate dibasic | — | 0.5 | — | — |
| Citric acid | 0.2 | — | — | — |
| Sodium citrate | 0.2 | — | — | — |
| Hydrochloric acid | q.s. | q.s. | — | — |
| Water For Injection | q.s. | q.s. | q.s. | q.s. |

Procedure: a) collect a suitable vehicle (such as water for injection) in a suitable container; b) cool the vehicle of step a) to a suitable temperature range (such as 20 to 25° C.); c) add suitable excipients (such as a wetting agent, tonicity adjusting agent) with stiffing for a suitable time to produce a clear solution; d) add the drug to the solution of step c) with stiffing for a suitable time; e) stir the solution of step d) for a suitable time and the adjust pH using one or more suitable pH adjusting agents; f) make the volume up to final batch size using water for injection; g) filter the solution of step f) using suitable filters (such as 0.2-micron size); h) fill the solution of step g) in appropriate size vials; i) stopper the vials using rubber stoppers; j) cap the vials using suitable seals (such as aluminum flip-off seals).

What is claimed:

1. A pharmaceutical composition for parenteral administration comprising:
(a) vibegron or its pharmaceutically acceptable salts, esters, solvates, polymorphs, enantiomers, or mixtures thereof,
(b) a pharmaceutically acceptable carrier, and
(c) one or more pharmaceutically acceptable excipients,
wherein the composition has a pH of about 3 to about 9.5.

2. The pharmaceutical composition of claim 1, wherein the parenteral administration is by subcutaneous injection or by intramuscular injection.

3. The pharmaceutical composition of claim 1, wherein the composition is an immediate release composition.

4. The pharmaceutical composition of claim 1, wherein the carrier is an aqueous vehicle, water-miscible solvent, non-aqueous vehicle, or a mixture of aqueous and non-aqueous vehicle.

5. The pharmaceutical composition of claim 1, wherein the one or more pharmaceutically acceptable excipients are selected from the group comprising preservatives, diluents, solvents, tonicity adjusting agents, pH adjusting agents, chelating agents, wetting agents, and antioxidants.

6. A process for the preparation of a pharmaceutical composition for parenteral administration of vibegron of claim 1 comprising the steps of:
(a) preparing a mixture comprising vibegron, carrier, and one or more pharmaceutically acceptable excipients.

7. The process of claim 6, wherein the process comprises one or more additional steps selected from the group consisting of (b) purging the mixture with an inert gas; (c) filtering the mixture through a filter; (d) filling the mixture into a suitable container; and (e) autoclaving the mixture at elevated temperature and elevated pressure.

8. The pharmaceutical composition of claim 1, wherein the composition comprises from about 0.01% to about 40% of vibegron or its pharmaceutically acceptable salts, esters, solvates, polymorphs, enantiomers, or mixtures thereof,

9. The pharmaceutical composition of claim 5, wherein the preservatives are selected from the group comprising benzyl alcohol, chlorobutanol, 2-ethoxyethanol, m-cresol, chlorocresol, benzalkonium chloride, benzethonium chloride, benzoic acid, sorbic acid, chlorhexidine, thimerosal, methyl paraben, propyl paraben, phenyl mercuric acetate, phenyl mercuric nitrate, and combinations thereof.

10. The pharmaceutical composition of claim 5, wherein the tonicity adjusting agents are selected from the group comprising sodium chloride, sodium sulfate, dextrose, mannitol, sorbitol, glycerol, potassium chloride, glycerin, lactose, trehalose, ammonium carbonate, ammonium chloride, ammonium lactate, ammonium nitrate, ammonium phosphate, ammonium sulfate, ascorbic acid, bismuth sodium tartrate, boric acid, calcium chloride, disodium calcium edetate, calcium gluconate, calcium lactate, citric acid, dextrose, diethanolamine, dimethyl sulfoxide, disodium edetate, trisodium edetate monohydrate, sodium fluorescein, fructose, galactose, lactic acid, lactose, magnesium chloride, magnesium sulfate, polyethylene glycol, potassium acetate, potassium chlorate, potassium chloride, potassium iodide, potassium nitrate, potassium phosphate, potassium sulfate, propylene glycol, sodium acetate, sodium bicarbonate, sodium biphosphate, sodium bisulfate, sodium borate, sodium bromide, sodium cacodylate, sodium carbonate, sodium chloride, sodium citrate, sodium iodide, sodium lactate, metabisulfate sodium sulfite, sodium nitrate, sodium nitrite, sodium phosphate, sodium propionate, sodium succinate, sodium sulfite, sodium tartrate, sodium thiosulfate, sorbitol, maltose, sucrose, tartaric acid, triethanolamine, urea, urethane, uridine zinc sulfate, zinc chloride, albumin, amino acid, and combinations thereof.

11. The pharmaceutical composition of claim 5, wherein the pH adjusting agents are selected from the group comprising inorganic acids, organic acids, inorganic bases, and organic bases, borate buffers, tartarate buffers, lactate buffers, citrate buffers, phosphate buffers, citric acid/phosphate buffers, carbonate/carbonic acid buffers, succinate/succinic acid buffers, ammonium buffers.

12. The pharmaceutical composition of claim 5, wherein the chelating agents are selected from the group comprising ethylenediamine tetracetic acid salts, diethylenetriaminepentaacetic acid, ethylene glycol-bis(β-aminoethyl ether)-tetraacetic acid, N-(hydroxy ethyl) ethylenediaminetriacetic acid, 8-hydroxyquinoline, citric acid, tartaric acid, phosphoric acid, gluconic acid, and combinations thereof.

13. The pharmaceutical composition of claim 5, wherein the antioxidants are selected from the group comprising ascorbic acid and its derivatives, thiol derivatives, tocopherols, butylated hydroxyanisole, butylated hydroxytoluene, sulfurous acid salts, propyl gallate, and combinations thereof.

14. The pharmaceutical composition of claim 5, wherein the wetting agents are selected from the group comprising anionic, cationic, zwitterionic, and non-ionic surfactants.

15. The pharmaceutical composition of claim 1, wherein the composition is provided in a kit comprising:
(a) a parenteral composition of vibegron or its pharmaceutically acceptable salts, esters, solvates, polymorphs, enantiomers, or mixtures thereof in a suitable container like a vial, ampoule, syringe, auto-injector, and a single or multi-compartment pen,
(b) optionally a container comprising a carrier for preparing the composition, and
(c) instructions for preparing and administration of the composition.

16. A method for treating a condition selected from the group comprising double chin disorder, benign symmetric lipomatosis, adiposis dolorosa, lipedema, familial partial lipodystrophy, and localized fat, the method comprising administering to the subject a composition according to claim 1.

17. The method of claim 16, wherein the therapeutically effective amount of vibegron or its pharmaceutically acceptable salts, esters, solvates, polymorphs, enantiomers, or mixtures thereof is administered within a plurality of treatment sessions.

18. A method of treating double chin disorder, benign symmetric lipomatosis, adiposis dolorosa, lipedema, familial partial lipodystrophy or localized fat in a subject, the method comprising parenteral administration to the subject of a pharmaceutical composition comprising:
(a) vibegron or its pharmaceutically acceptable salts, esters, solvates, polymorphs, enantiomers, or mixtures thereof,
(b) a pharmaceutically acceptable carrier, and
(c) one or more pharmaceutically acceptable excipients.

19. A pharmaceutical composition for parenteral administration comprising:
(a) vibegron or its pharmaceutically acceptable salts, esters, solvates, polymorphs, enantiomers, or mixtures thereof,
(b) a preservative,
(c) a pH adjusting agent,
(d) a tonicity adjusting agent,
(e) optionally an antioxidant, a chelating agent and a wetting agent, and
(f) a carrier.

20. The pharmaceutical composition of claim 19, wherein the composition comprises:
(a) about 0.01% to about 40% of vibegron or its pharmaceutically acceptable salts, esters, solvates, polymorphs, enantiomers, or mixtures thereof,
(b) about 0.01% to about 7% of preservative,
(c) about 0.01% to about 8% of pH adjusting agent,
(d) about 0.1% to about 40% of a tonicity adjusting agent,
(e) optionally about 0.01% to about 7% of antioxidant, about 0.01% to about 4% of chelating agent and about 0.01% to about 2% of wetting agent, and
(f) about 20% to about 99.99% of a pharmaceutically acceptable carrier.
